(19) United States
(12) Patent Application Publication
(10) Pub. No.: US 2008/0317692 A1
(43) Pub. Date: Dec. 25, 2008

(54) SUICIDE SUBSTRATES OF TYROSINASE AND USE THEREOF

(75) Inventors: Te-Sheng Chang, Tainan (TW); Ching-Yi Wu, Tainan (TW)

Correspondence Address: WPAT, PC;INTELLECTUAL PROPERTY ATTORNEYS, 2030 MAIN STREET, SUITE 1300, IRVINE, CA, 92614, US

(73) Assignees: TE-SHENG CHANG, Tainan (TW); MON-HAN WU, Tainan (TW); SUSTINEO BIOTECHNOLOGY CO., LTD, Tainan (TW)

(21) Appl. No.: 11/768,122

(22) Filed: Jun. 25, 2007

(51) Int. Cl. A61K 31/352; A61K 8/33; A61Q 19/02; C07D 311/22

(52) U.S. Cl. 424/63; 514/456; 549/399

(57) ABSTRACT

The present invention provides a novel compound and a method of using the compound to inactivate tyrosinase activity in a subject comprising administering the patient with an effective amount of this novel compound.

### FIELD OF THE INVENTION

This invention relates to a composition comprising a compound for inactivating tyrosinase. This invention also relates to a method of inactivating tyrosinase activity.

### DESCRIPTION OF PRIOR ART

Tyrosinase (EC 1.14.18.1) is a copper-containing monooxygenase widely distributed in nature. The structures of model tyrosinases have been elucidated (Klabunde, T. et al., 1998, Nat. Struct. Biol., 5, 1084-1090.; Matoba, Y. et al., 2006, J. Biol. Chem., 281, 8981-8990.). The enzyme catalyzes the first two reactions of melanin synthesis, the hydroxylation of L-tyrosine to 3,4-dihydroxyphenylalanine, L-DOPA, and the oxidation of L-DOPA to dopaquinone. This o-quinone is a highly reactive compound and can polymerize spontaneously to form melanin (Seo, S. Y. et al., 2003, J. Agric. Food Chem., 51, 2837-2853.). The enzyme is also known as a polyphenol oxidase (PPO) and is responsible for enzymatic browning reactions in damaged fruits during post harvest handling and processing, which is caused by the oxidation of phenolic compounds in the fruits. Both the hyperpigmentation in skin and the enzymatic browning in fruits are not desirable, and inhibiting the tyrosinase activity has been the subject of many studies (Baurin, N. et al., 2002, J. Ethnopharmacol., 82, 155-158.; Chen, Q. et al., 2002, J. Agric. Food Chem., 50, 4108-4112.; Kim, Y. M. et al., 2002, J. Biol. Chem., 277, 16340-16344.; Shiino, M. et al., 2003, Bioorg. Chem., 31, 129-135.). There is a concerted effort to search for naturally occurring tyrosinase inhibitors from plants, because plants constitute a rich source of bioactive chemicals and many of them are largely free from harmful adverse effects (Lee, G. C. et al., 1997, Food Chem., 60, 231-235.).

Suicide inactivation of tyrosinase has been reported in early studies. Despite suicide substrates of tyrosinase are useful as skin-depigmenting and food-antibrowning agents; potent suicide substrates have rarely been discovered.

### SUMMARY OF THE INVENTION

The present invention provides a composition comprising a compound of formula

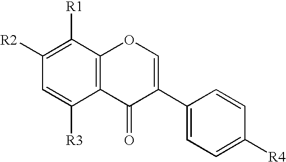

wherein

R₁, R₂, R₃, or R₄ is H, hydroxyl, or its esterized or glycosylated or alkylated derivatives.

The present invention also provides a method of inactivating tyrosinase activity in a subject comprising administering the patient with an effective amount of a compound of formula

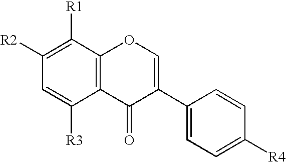

wherein

R₁, R₂, R₃, or R₄ is H, hydroxyl, or its esterized or glycosylated or alkylated derivatives.

### BRIEF DESCRIPTION OF THE DRAWINGS

FIG. 1 shows chemical structures of investigated compounds.

FIG. 2 shows time course of tyrosinase reaction (⋄) inhibited by 0.05 mM 7,8,4′-trihydroxyisoflavone (▴) or 5,7,8,4′-tetrahydroxyisoflavone (▪) with mushroom tyrosinase (100 units/mL). L-Tyrosine (A) or L-DOPA (B) at 0.1 mM was used as the substrate.

FIG. 3 shows HPLC chromatograms of the reaction mixture containing 100 μM 5,7,8,4′-tetrahydroxyisoflavone and tyrosinase (1000 units/mL, A-C) or heat-denatured tyrosinase (1000 units/mL, D) in 1 mL of 50 mM phosphate buffer (pH 6.8). Samples were collected at 0 sec (A), 10 sec (B), 30 sec (C), and 10 sec (D).

FIG. 4 shows inhibitory effects of 7,8,4′-trihydroxyisoflavone (3 μM, Δ; 10 μM, ▴) and 5,7,8,4′-tetrahydroxyisoflavone (3 μM, □; 10 μM, ▪) on mushroom tyrosinase activity (⋄) with various durations of preincubation.

FIG. 5 shows titration of mushroom tyrosinase with either 7,8,4′-trihydroxyisoflavone (A) or 5,7,8,4′-tetrahydroxyisoflavone (B). The enzyme (0.1 μM) and the isoflavone (0.55-7.7 μM in panel A and 0.1-3.5 μM in panel B) were preincubated in 1 mL of 50 mM phosphate buffer (pH 6.8) at 25° C. for 30 min.

FIG. 6 shows determination of Michaelis constants and maximal inactivation rate constants of 7,8,4′-trihydroxyisoflavone (A) or 5,7,8,4′-tetrahydroxyisoflavone (B).

FIG. 7 illustrates the stability test on esterized 7,8,4′-Trihydroxyisoflavone and 5,7,8,4′-Tetrahydroxyisoflavone.

### DETAILED DESCRIPTION OF THE INVENTION

The two isoflavones 7,8,4′-trihydroxyisoflavone and 5,7,8,4′-tetrahydroxyisoflavone, in the present invention, were proven to be potent and unique suicide substrates of mushroom tyrosinase with low partition ratios, low Michaelis constants, and high maximal inactivation rate constants. It is worthwhile to further apply these two suicide substrates in the cosmetics and medical industry.

### Identification of 7,8,4′-Trihydroxyisoflavone and 5,7,8,4′-Tetrahydroxyisoflavone as Suicide Substrates of Mushroom Tyrosinase

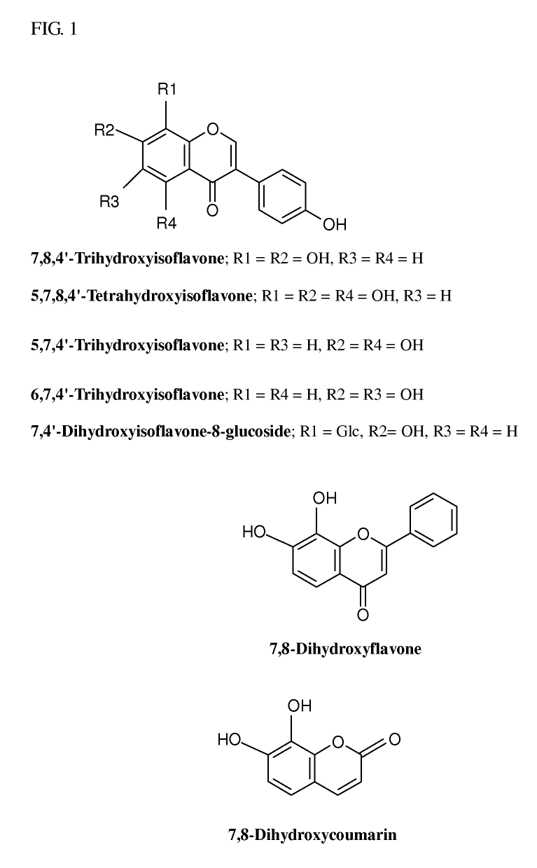

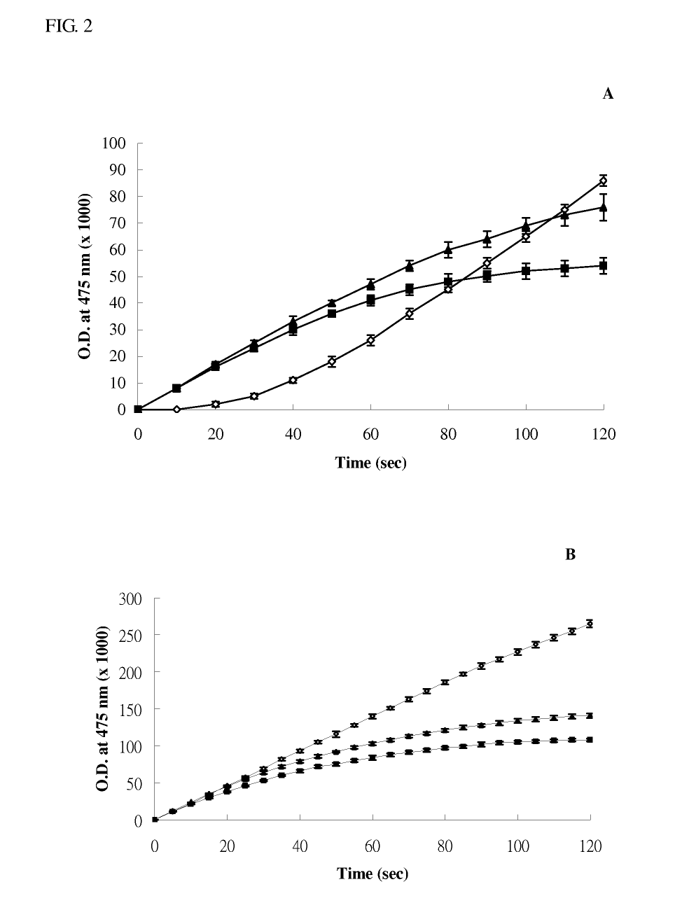

To study the tyrosinase inhibition by 7,8,4′-trihydroxyisoflavone and 5,7,8,4′-tetrahydroxyisoflavone (FIG. 1), the inhibitory effects of the two isoflavones on both monophenolase and diphenolase activities of mushroom tyrosinase were examined. The results are shown in FIG. 2. When the enzymatic reaction was carried out with L-tyrosine as a substrate, a marked lag time, characteristic of monophenolase activity, was observed, simultaneously with the appearance of dopachrome (FIG. 2A). The lag time is the time required to reach the steady-state concentration of o-diphenol. The length of the lag time can be shortened or abolished by the presence of reducing agents or o-diphenol substrates, such as L-DOPA. As can be seen from FIG. 2A, L-tyrosine was oxidized by the enzyme without the lag time in the presence of each of the two isoflavones. Because the two compounds contain an o-diphenol structure, the result implied that the two isoflavones might act as substrates of mushroom tyrosinase. In this situation, the met form of the enzyme, which is the major form in the enzyme resting state, was quickly reduced to its deoxy form by catalyzing the o-diphenol substrates. Then the deoxy form of tyrosinase spontaneously is converted to its oxy form, which is the only form that could bind with L-tyrosine. On the other hand, the two isoflavones slowed and stopped the formation of dopachrome when L-tyrosine was used as a substrate, behaving, therefore, as an inhibitor of the monophenolase activity of mushroom tyrosinase.

Furthermore, when the diphenolase activity of tyrosinase was examined by using L-DOPA as a substrate, the reaction immediately reached a steady state (FIG. 2B). The presence of each of the two isoflavones in the assay medium resulted in reduction in the diphenolase activity (FIG. 2B). The above results revealed that the two isoflavones inhibited both monophenolase and diphenolase activities of mushroom tyrosinase.

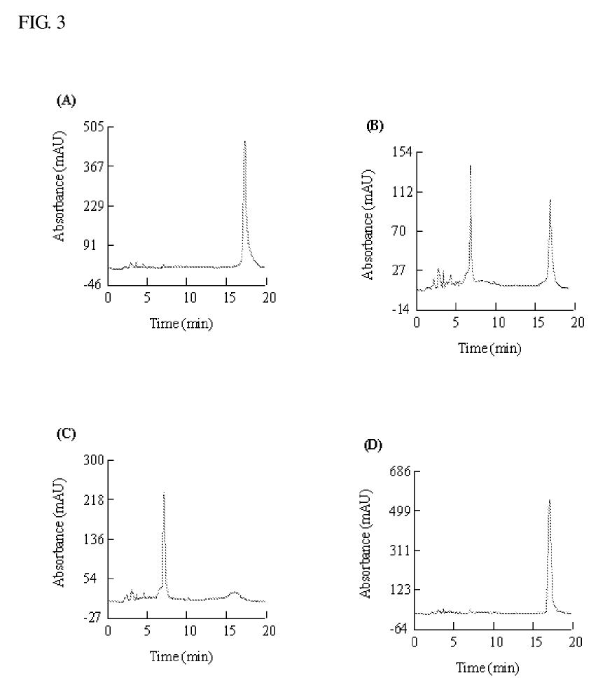

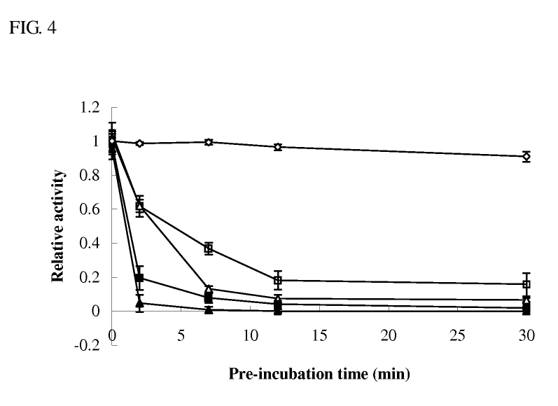

To ascertain whether the two isoflavones behaved as the substrates of mushroom tyrosinase, the enzymatic reactions of tyrosinase with 5,7,8,4′-tetrahydroxyisoflavone and 7,8,4′-trihydroxyisoflavone were studied by mixing the isoflavone and tyrosinase in phosphate buffer at pH 6.8. The reaction mixture was analyzed by HPLC, and the results are shown in FIG. 3. The isoflavone (t_{R}=17.3 min) started to decrease and a new peak (t_{R}=7.1 min) gradually appeared during the catalytic reaction with active tyrosinase. In contrast, the isoflavone remained constant during the catalytic reaction with the heat-denatured tyrosinase. A similar result was also obtained with 7,8,4′-trihydroxyisoflavone as the substrate (data not shown). The results revealed that the two isoflavones acted as the substrates of mushroom tyrosinase. The above results showed that the two compounds possess the characteristics of both a substrate and an inhibitor for mushroom tyrosinase. It is known that tyrosinase could be irreversibly inhibited by its o-diphenol substrates, such as L-DOPA and catechol. These substrates were also named as suicide substrates or mechanism-based inhibitors. We therefore investigated further whether 7,8,4′-trihydroxyisoflavone and 5,7,8,4′-tetrahydroxyisoflavone could irreversibly inhibit tyrosinase. The results are shown in FIG. 4. The enzyme activity in the preincubation mixture without the addition of the two isoflavones remained constant during 30 min of reaction. However, preincubation of tyrosinase with each of the two isoflavones quickly inactivated the enzyme within the first 2 min of preincubation. With the addition of 10 μM isoflavone in the preincubation mixture, the enzyme was totally inactivated after 7 min of preincubation. Moreover, the enzyme activity in the preincubation mixture was not restored by using dialysis or molecular exclusion chromatography to remove compounds of low molecular weight such as the two isoflavones (data not shown). From these results, 7,8,4′-trihydroxyisoflavone and 5,7,8,4′-tetrahydroxyisoflavone were identified as irreversible inhibitors and they belonged to suicide substrates or mechanism-based inhibitors for mushroom tyrosinase.

Therefore, the present invention also provided a method of inactivating tyrosinase activity in a subject comprising administering the patient with an effective amount of a compound of formula

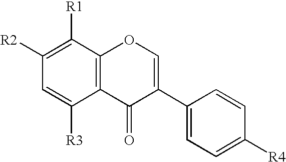

wherein R₁, R₂, R₃, or R₄ is H, hydroxyl, or its esterized or glycosylated or alkylated derivatives. In the present of the invention, the preferred compound wherein the R₁, R₂ and R₄ are hydroxy or; the preferred compound wherein the R₁, R₂, R₃ and R₄ are hydroxy.

In addition, the lipase is used to stabilize the said compound by esterification. Through the above reaction, the esterified compounds became suitable for active ingredients of cosmetics and were able to be applied to whiten skin of human being who suffers hyperpigmentation in skin.

In the preferred embodiment, the effective amount of 7,8,4′-tetrahydroxyisoflavone is 0.1-8.0 μM based on 0.1M of tyrosinase; the prefer effective amount of 7,8,4′-tetrahydroxyisoflavone is 0.55-7.7 μM based on 0.1 μM of tyrosinase. The effective amount of 5,7,8,4′-tetrahydroxyisoflavone is 0.1-3.5 μM based on 0.1 μM of tyrosinase

### Determination of Partition Ratios of the Two Suicide Substrates.

An initial step, which is of prime importance in every quantitative work with suicide substrates, is to determine the molar proportion for inactivation, that is, the number of molecules of inhibitors required to completely inactivate one molecule of the enzyme. The mechanism of suicide substrate has been extensively studied by Waley, who proposed a simple branched reaction pathway as follows, in which an intermediate Y may give either active enzyme and product or inactive enzyme.

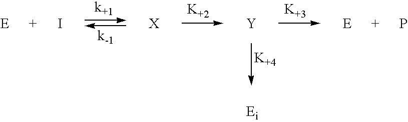

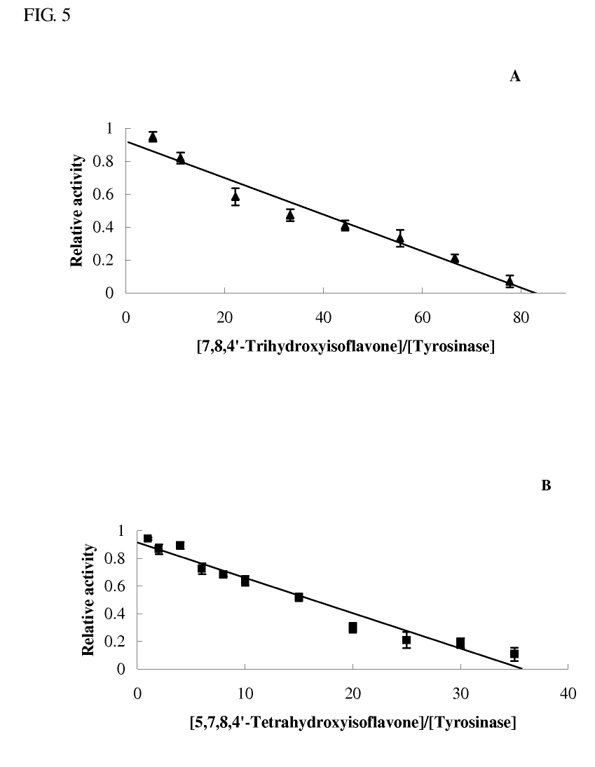

In the above scheme, E and Eᵢ are enzyme and inactivated enzyme, respectively; P is product; X is the first intermediate and Y is another intermediate. The intermediate Y has a choice of reaction, governed by the partition ratio r, where r=k₊₃/k₊₄. The molar proportion for inactivation, as defined above, may be determined by plotting the fractional activity remaining against the ratio of the initial concentration of inhibitor to that of enzyme. The intercept on the abscissa is 1+r in the plot, when r>1. The result is shown in FIG. 5. When tyrosinase was preincubated with each of varied amounts of 7,8,4′-trihydroxyisoflavone or 5,7,8,4′-tetrahydroxyisoflavone, the fractional activity remaining was proportional to the molar ratio of the added isoflavone to enzyme. By extrapolation, 82.7±5.9 and 36.5±3.8 molecules of 7,8,4′-trihydroxyisoflavone and 5,7,8,4′-tetrahydroxyisoflavone, respectively, were required to inactivate 1 molecule of the enzyme. When less than 83 and 37 molar proportions of 7,8,4′-trihydroxyisoflavone and 5,7,8,4′-tetrahydroxyisoflavone, respectively, were used, the suicide reaction ceased because all of the suicide substrates had been consumed, and the fractional activity remaining was not appreciably different when the duration of incubation was varied from a few minutes to a few hours. Therefore, the partition ratios of the two suicide substrates were calculated to be 81.7±5.9 and 35.5±3.8 for 7,8,4′-trihydroxyisoflavone and 5,7,8,4′-tetrahydroxyisoflavone, respectively, from the intercept on the abscissa in FIG. 5, which is 1+r.

### Determination of Michaelis Constant and Maximal Inactivation Rate Constant of the Two Suicide Substrates

The kinetics of inhibition of 7,8,4′-trihydroxyisoflavone and 5,7,8,4′-tetrahydroxyisoflavone were studied by using the method of Frere et al. and by measuring the oxidation of L-DOPA by mushroom tyrosinase in the presence of each of the two suicide substrates. During the assay, the concentration of the tested suicide substrate was much higher than that of the enzyme. When the progress of the inactivation of the enzyme was monitored at high suicide substrate/enzyme ratios, the concentration of suicide substrate ([I]) could be considered as constant throughout the process and a pseudo-first-order rate constant (kᵢ) could be determined by using equation 1, where kᵢ₋ₘₐₓ and K_{I} represent the maximal inactivation velocity and the Michaelis constant of the inactivator.

$${k}_{i} = \frac{{k}_{i - max} [ I ]}{[ I ] + {K}_{I}} \qquad ( 1 )$$

In the presence of L-DOPA, the exponential decrease in the rate of oxidation of L-DOPA gave an apparent first-order rate constant k_{obs}, which was computed from plots of ln(vₜ/v₀) against t, where v₀ and vₜ are the rates of increase of absorbance at 475 nm, at zero time and at time t, respectively. Assuming a competitive interaction between the added isoflavone and L-DOPA with the enzyme, the variation of k_{obs} with the concentrations of the isoflavone ([I]) and L-DOPA ([S]) are given by equation 2

$${k}_{obs} = \frac{{k}_{i - max} [ I ]}{[ I ] + 9 {K}_{I} ( 1 + [ S ] / {K}_{S} )} \qquad ( 2 )$$

where K_{S} and K_{I} are the Michaelis constants for L-DOPA and the isoflavone, respectively. When equation 2 is written as

[I]/k_{obs}=[I]/kᵢ₋ₘₐₓ+K_{I}/kᵢ₋ₘₐₓ(1+[S]/K_{S}) (3)

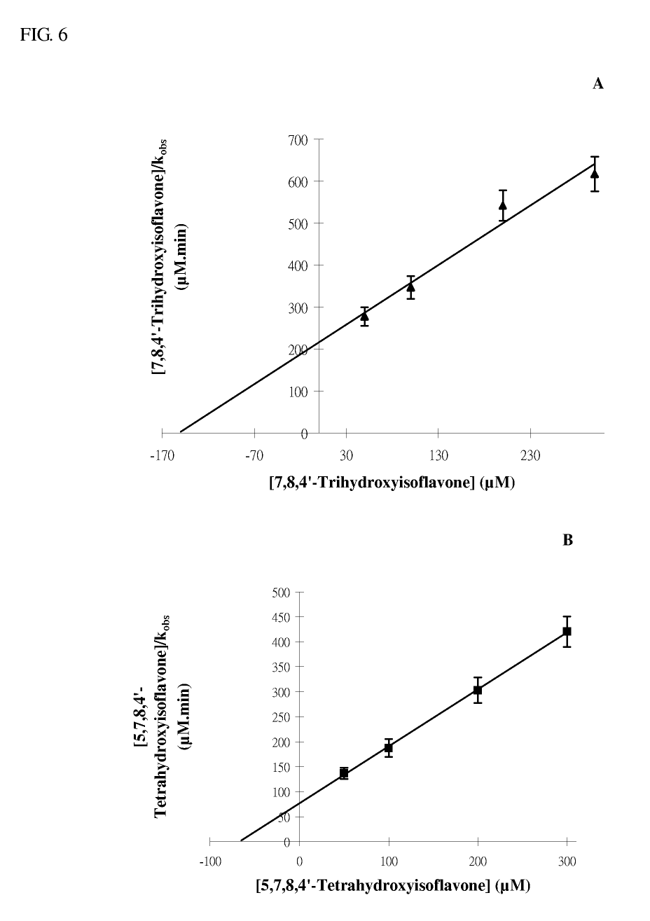

it is clear that a plot of [I]/k_{obs} against [I] will be linear and that kᵢ₋ₘₐₓ and K_{I} can be found from the intercept and slope. The result is shown in FIG. 6. Under the same experimental conditions, K_{S} was determined to be 0.25±0.01 mM. Hence, the values of kᵢ₋ₘₐₓ and K_{I} were calculated to be 0.79±0.08 and 1.01±0.04 min-land 18.70±2.31 and 7.81±0.05 μM for 7,8,4′-trihydroxyisoflavone and 5,7,8,4′-tetrahydroxyisoflavone, respectively, when L-DOPA was used as the enzyme substrate. The high kᵢ₋ₘₐₓ and low K_{I} values of the two suicide substrates meant the second-order rate constants (kᵢ₋ₘₐₓ/K_{I}) were large. Thus, the two isoflavones are “high-reactivity, high-affinity” suicide substrates of mushroom tyrosinase.

### Structure Analysis on the Potency of Suicide Substrate

To verify the relationship between the chemical structure and the potency of suicide substrate of mushroom tyrosinase, we used irreversible inhibitory ability as a primary guide. The tested structural analogues of 7,8,4′-trihydroxyisoflavone included 5,7,4′-trihydroxyisoflavone, 6,7,4′-trihydroxyisoflavone, 7,4′-dihydroxyisoflavone-8-glucoside, 7,8-dihydroxycoumarin, and 7,8-dihydroxyflavone (FIG. 1). The irreversible inhibitory assay was conducted by the reaction of preoccupation of the compound with mushroom tyrosinase. As a consequence, we found that none of the tested analogous compounds irreversibly inhibited diphenolase activity of mushroom tyrosinase. Hence, it was clear that when the hydroxyl group at the C8 position of the A-ring in 7,8,4′-trihydroxyisoflavone was exchanged with that at the C5 (5,7,4′-trihydroxyisoflavone) or C6 (6,7,4′-trihydroxyisoflavone) position or exchanged with the glucoside (7,4′-dihydroxyisoflavone-8-glucoside), the potency of the irreversible inhibitory activity totally disappeared. This indicated the 7,8-dihydroxyl groups in both 7,8,4′-trihydroxyisoflavone and 5,7,8,4′-tetrahydroxyisoflavone played an important role in the suicide nature of the substrate for mushroom tyrosinase. On the other hand, when the isoflavone skeleton was replaced with that of flavone (7,8-dihydroxyflavone) or coumarin (7,8-dihydroxycoumarin), the irreversible inhibitory activity also disappeared, even when the two hydroxyl groups at the C7 and C8 positions in the A-ring were maintained. From the above results, it was thus concluded that not only the 7,8-dihydroxyl groups but also the isoflavone skeleton in both 7,8,4′-trihydroxyisoflavone and 5,7,8,4′-tetrahydroxyisoflavone were absolutely necessary for the compounds to function as potent suicide substrates of mushroom tyrosinase. The results also revealed that the two suicide substrates are unique.

In addition, the lipase is used to stabilize the said compound by esterification. Through the above reaction, the esterified compounds became suitable for active ingredients of cosmetics and were able to be applied to whiten skin of human being who suffers hyperpigmentation in skin.

In the preferred embodiment, the effective amount of 7,8,4′-tetrahydroxyisoflavone is 0.1-8.0 μM based on 0.1M of tyrosinase; the prefer effective amount of 7,8,4′-tetrahydroxyisoflavone is 0.55-7.7 μM based on 0.1 M of tyrosinase. The effective amount of 5,7,8,4′-tetrahydroxyisoflavone is 0.1-3.5 μM based on 0.1 μM of tyrosinase

### Modification and Protection of 7,8,4′-Trihydroxyisoflavone, and 5,7,8,4′-Tetrahydroxyisoflavone

In the practical use of 7,8,4′-trihydroxyisoflavone and 5,7,8,4′-tetrahydroxyisoflavone in cosmetic products, the functional groups (such as hydroxy) of 7,8,4′-trihydroxyisoflavone and 5,7,8,4′-tetrahydroxyisoflavone can be protected by known methods (such as the use of esterification or glycosylation) to form related derivatives. Such derivatives can be added in the commercial products. When such derivatives are entered into cells, they would be hydrolyzed to actives 7,8,4′-trihydroxyisoflavone and 5,7,8,4′-tetrahydroxyisoflavone by enzymes (such as lipase or glycolytic enzymes) within cells. Such protection for these compounds avoids them from oxidization in a product, enhances stability and decreases skin irritation.

### EXAMPLE

The examples below are non-limiting and are merely representative of various aspects and features of the present invention.

### Example 1

### Materials

Mushroom tyrosinase (2870 units/mg), L-tyrosine, L-DOPA, dimethyl sulfoxide (DMSO), and 5,7,4′-trihydroxyisoflavone (genistein) were purchased from Sigma Chemical Co. (St. Louis, Mo.). One unit of mushroom tyrosinase is defined as the amount of the enzyme that could induce 0.001 ΔA₂₈₀ per min at pH 6.5 at 25° C. in 3 mL of reaction mixture containing 1-tyrosine. 7,4′-Dihydroxyisoflavone-8-glucoside (puerarin) was obtained from Fluka Chemical Co. (Buchs, Switzerland). 7,8-Dihydroxycoumarin and 7,8-dihydroxyflavone were from Tokyo Chemical Industry Co. (Tokyo, Japan). High-performance liquid chromatography (HPLC) grade acetonitrile and acetic acid were from J. T. Baker (Phillipsburg, N.J.). Other reagents and solvents used were of analytical grade and were used as received.

### Example 2

### Isolation of 6,7,4′-Trihydroxyisoflavone, 7,8,4′-Trihydroxyisoflavone, and 5,7,8,4′-Tetrahydroxyisoflavone from Soygerm Koji

The purification process of 6,7,4′-trihydroxyisoflavone, 7,8,4′-trihydroxyisoflavone, and 5,7,8,4′-tetrahydroxyisoflavone in soygrem koji was carried out by using the anti-tyrosinase activity assay as a guide. Soygerm koji (500 g) was refluxed with 5 L of methanol for 3 h to give a methanol extract (102 g). The extract was suspended in water (0.1 L) and re-extracted with hexane and ethyl acetate. Each solute fraction was concentrated under vacuum to give hexane (54 g), ethyl acetate (5.43 g), and water (37 g) fractions. The ethyl acetate fraction (100 mg/mL in DMSO) showed the highest anti-tyrosinase activity (IC₅₀=0.19 mg/mL). The ethyl acetate extract was then fractionated by silica gel column chromatography (50×2.6 cm i.d.) with 0.5 L each of hexane/ethyl acetate (3:1), hexane/ethyl acetate (1:1), ethyl acetate, ethyl acetate/methanl (1:1), and methanol as eluents. The ethyl acetate fraction showed strongest anti-tyrosinase activity and was purified by repeated HPLC using a 250×10 mm i.d., ODS 2 Spherisorb semipreparative C18 reversed-phase column (Phase Separation Ltd., Deeside Industrial Park, Clwyd, U.K.). The gradient elution using water (A) containing 0.1% (v/v) acetic acid and acetonitrile (B) consisted of an isocratic elution for 10 min with 14% B and a linear gradient for 50 min with 20% to 40% B at a flow rate of 3 mL/min. The elution of the peaks was collected, dried, and assayed for anti-tyrosinase activity. The chemical structures of purified 6,7,4′-trihydroxyisoflavone, 7,8,4′-trihydroxyisoflavone, and 5,7,8,4′-tetrahydroxyisoflavone were identified by mass and NMR spectrometry.

### Example 3

### Instrumental Analyses of 6,7,4′-Trihydroxyisoflavone, 7,8,4′-Trihydroxyisoflavone, and 5,7,8,4′-Tetrahydroxyisoflavone

¹H NMR spectra were recorded with a Varian Gemini NMR spectrometer at 400 MHz and ¹³C NMR spectra with a Varian Gemini NMR spectrometer at 100 MHz in DMSO. FAB MS spectra were obtained with a JEOL TMSD-100. The physicochemical properties of 6,7,4′-trihydroxyisoflavone, 7,8,4′-trihydroxyisoflavone, and 5,7,8,4′-tetrahydroxyisoflavone are given next. 6,7,4′-Trihydroxyisoflavone: ¹H NMR (DMSO-d₆), δ 6.78 (2H, d, J=8.8 Hz, H-3′, 5′), 6.84 (1H, s, H-8), 7.34 (2H, d, J=8.8 Hz, H-2′, 6 ), 7.36 (1H, s, H-5), 8.21 (1H, s, H-2), 9.57 (3H, br s, OH-6,7,4′); ¹³C NMR (DMSO-d₆), δ 174.8 (C-4), 157.3 (C-4′), 152.8 (C-7), 152.6 (C-2), 151.2 (C-9), 145.0 (C-6), 130.4 (C-2′, 6′), 123.2 (C-1′), 123.1 (C-3), 116.9 (C-10), 115.3 (C-3′, 5′), 108.4 (C-5), 103.0 (C-8). FAB MS, m/z 271 [M+H]⁺.

7,8,4′-Trihydroxyisoflavone: ¹H NMR (DMSO-d₆), δ 6.79 (2H, d, J=8.3 Hz, H-3′, 5′), 6.94 (1H, d, J=8.7 Hz, H-6), 7.37 (2H, d, J=8.3 Hz, H-2′, 6′), 7.45 (1H, d, J=8.7 Hz, H-5), 8.30 (1H, s, H-2), 9.46 (1H, br s, OH-7), 9.58 (1H, br s, OH-4′), 10.37 (1H, br s, OH-8); ¹³C NMR (DMSO-d₆), } 175.6 (C-4), 157.4 (C-4′), 153.0 (C-2), 150.2 (C-7), 147.0 (C-9), 133.2 (C-8), 130.4 (C-2′, 6′), 123.2 (C-1′), 123.0 (C-3), 117.7 (C-10), 116.0 (C-5), 115.3 (C-3′, 5′), 114.5 (C-6); FAB MS, m/z 271 [M+H]⁺.

5,7,8,4-Tetrahydroxyisoflavone: ¹H NMR (DMSO-d₆), δ 6.29 (1H, s, H-6), 6.81 (2H, d, J=9.0 Hz, H-3′, 5′), 7.36 (2H, d, J=9.0 Hz, H-2′, 6′), 8.31 (1H, s, H-2), 8.86 (1H, br s, OH-7), 9.70 (1H, br s, OH-4′), 10.71 (1H, br s, OH-8); ¹³C NMR (DMSO-d6), δ 180.5 (C-4), 157.2 (C-4′), 153.8 (C-2), 153.3 (C-5), 153.0 (C-7), 145.7 (C-9), 130.1 (C-2′, 6′), 124.8 (C-8), 121.7 (C-1′), 121.3 (C-3), 115.0 (C-3′, 5′), 103.9 (C-10), 98.6 (C-6); FAB MS, m/z 287 [M+H]⁺.

### Example 4

### Enzymatic Assay of Tyrosinase

Ten microliters of the test sample (dissolved in DMSO) was mixed with 970 μL of 0.112 mM substrate (L-tyrosine or L-DOPA dissolved in 50 mM phosphate buffer, pH 6.8) at 25° C. for 2 min. Then, 20 μL of tyrosinase (1000 units/mL in phosphate buffer) was added to initiate the reaction. The increase in absorbance at 475 nm due to the formation of dopachrome was monitored with a spectrophotometer.

In irreversible inhibitory activity assays, 20 units of tyrosinase was preincubated with a 3 or 10 μM concentration of the tested isoflavone (dissolved in DMSO) in 1 mL of 50 mM phosphate buffer (pH 6.8) at 25° C. At intervals of 0, 2, 7, 12, and 30 min, 200 μL of the preincubation mixture was mixed with 800 μL of 2.5 mM L-DOPA and incubated at 25° C. for 10 min. The formation of dopachrome in each reaction was monitored with a spectrophotometer. The relative activity was calculated by dividing the absorbance at 475 nm of each reaction by that of the control reaction, in which DMSO replaced the added isoflavone. For recovery experiments, the preincubation mixture incubated for 30 min was either dialyzed twice against 200 mL of phosphate buffer at 4° C. for 1 h with stirring or centrifuged through a Sephadex G-25 spin column (Sigma). Then, the residual tyrosinase activities of the mixtures from the two treatments were assayed as described above.

The partition ratio of the suicide substrate was determined according to the method of Waley by incubating 500 μL of preincubation mixture containing 0.1 μM tyrosinase and 0.55-7.7 μM 7,8,4′-trihydroxyisoflavone or 0.1-3.5 μM 5,7,8,4′-tetrahydroxyisoflavone at 25° C. for 30 min. Then, 200 μL of preincubation mixture was mixed with 800 μL of 2.5 mM L-DOPA. The absorbance of the reaction mixture at 475 nm was monitored every 1 s with a spectrophotometer. The initial reaction velocities were measured from the slope at the first 2 min of the time course of the reaction curve. The relative activity of each reaction was calculated by dividing the initial velocity of the reaction with suicide substrate by that of the reaction without suicide substrate. The partition ratio of suicide substrate could be determined by plotting the fractional activity remaining against the ratio of the initial concentration of the suicide substrate to that of enzyme.

The Michaelis constants (K_{I}) and maximal inactivation rate constants (kᵢ₋ₘₐₓ) of suicide substrates were determined according to the method of Frere et al. (17). The inactivation reactions were carried out in the presence of 0.03 μM mushroom tyrosinase, 2.5 mM L-DOPA, and the suicide substrate at concentrations ranging from 50 to 300 μM, and the formation of dopachrome was monitored every second for 2 min with a spectrophotometer. Under these conditions, the rate of oxidation of L-DOPA progressively decreased and the apparent first-order rate constant (k_{obs}) for the inactivation was computed from the plots of ln(vₜ/v₀) against t, where v₀ and vₜ are the rates of increase of absorbance at 475 nm at zero time and at time t, respectively. Both K_{I} and kᵢ₋ₘₐₓ could thus be calculated, assuming a competition between the added isoflavone and L-DOPA.

For structure analysis of the two isoflavones on the inhibitory effects of mushroom tyrosinase, 20 units of tyrosinase was preincubated with the tested compound (10 μM for 7,8,4′-trihydroxyisoflavone and 5,7,8,4′-tetrahydroxyisoflavone; 100 μM for others) in 200 μL of 50 mM phosphate buffer (pH 6.8) at 25° C. for 30 min. Then, 800 μL of 2.5 mM L-DOPA was added, and the reaction mixture was incubated at 25° C. for 10 min. For comparison, another set of experiments was conducted by mixing immediately the tested compound, tyrosinase, and L-DOPA in 1 mL of phosphate buffer and incubated at 25° C. for 10 min. The formation of dopachrome in each reaction was monitored with a spectrophotometer. The relative activity was calculated by dividing the absorbance at 475 nm of each reaction mixture by that of the control reaction, in which DMSO replaced the tested compound. All enzymatic reactions described above were carried out at least three times independently, and the average values are presented.

### Example 5

### HPLC Analysis

HPLC analysis was performed on a Hitachi D-7000 HPLC (Hitachi, Ltd., Tokyo, Japan) system equipped with an L-7400 UV detector and a 250×4.6 mm i.d., ODS 2 Spherisorb C18 reversedphase column (Phase Separation Ltd.). The operating conditions were as follows: solvent, 30% acetonitrile/water containing 1% acetic acid; flow rate, 0.8 mL/min; detection, 262 nm; injected volume, 20 μL from a 1 mL assay system containing 100 μM isoflavone and 1000 units of mushroom tyrosinase in 50 mM phosphate buffer (pH 6.8).

### Example 6

### Esterification of Suicide Substrates and Purification of the Esterified Products

Reactions were conducted in 200 ml screw-caped glass vials. 2 mmol of 7,8,4′-Trihydroxyisoflavone, or 5,7,8,4′-Tetrahydroxyisoflavone was reacted with 4 mmol palmitic acid, in the presence of 0.68 g of Novozyme 435 immobilized-lipase and 1 g molecular sieve, and 15 ml acetone. The reactions were carried out in a thermostat shaker at 40° C. for 180 rpm and 24 hr. At the end of esterification reaction, the immobilized-lipase and molecular sieve were removed by filtration. The filtrates were collected and the acetone in them was evaporated under reduced pressure. The esterified products were recovered and purified by semi-preparative HPLC using a 250×10 mm i.d., ODS 2 Spherisorb semipreparative C18 reversed-phase column (Phase Separation Ltd., Deeside Industrial Park, Clwyd, U.K.). The elution used methanol/water/acetic acid (89.9:10:0.1;v/v) at a flow rate of 3 mL/min. The elution of the peaks was collected, dried, and assayed for stability activity.

### Example 7

### Stability Analysis

The purified esterified products and their original substrates were dissolved (10 mM) in the 50 mM of phosphate solution (pH 6.8). The reactions were stranded at 25° C. For each day, the samples were taken out for the analysis of the residue of each tested compounds. The decrease of the amount of the tested compounds in the solution was monitored by HPLC analysis.

### Example 8

### Stabilization of 7,8,4′-Trihydroxyisoflavone and 5,7,8,4′-Tetrahydroxyisoflavone

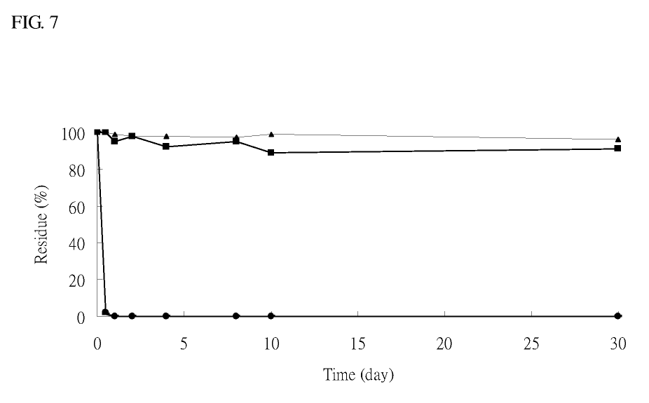

By using immobilized lipase, Novozyme 435, 7,8,4′-Trihydroxyisoflavone and 5,7,8,4′-Tetrahydroxyisoflavone were transferred to esterified 7,8,4′-Trihydroxyisoflavone and 5,7,8,4′-Tetrahydroxyisoflavone. At first 5,7,8,4′-Tetrahydroxyisoflavone, Novozyme435 and palmitic acid were mixed, sustained stirred, in methyl ethyl ketone solution buffer for 25 hours. After reacting, stabilization analysis was processed at different temperatures in air. 7,8,4′-Trihydroxyisoflavone was processed with the same procedure. The reacted two mixtures were placed at different temperature and HPLC was used to analyze the stability of 7,8,4′-Trihydroxyisoflavone, 5,7,8,4′-Tetrahydroxyisoflavone and their esterified products at different time points. FIG. 7 illustrated the result of stability test on 40° C. The result showed that esterified 7,8,4′-Trihydroxyisoflavone and 5,7,8,4′-Tetrahydroxyisoflavone were more stable than 7,8,4′-Trihydroxyisoflavone and 5,7,8,4′-Tetrahydroxyisoflavone. Accordingly, the esterified isoflavones were suitable for active ingredients of cosmetics.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

1. A composition comprising a compound of formula 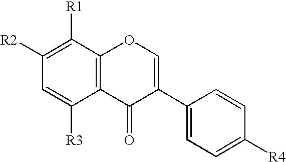
wherein R₁, R₂, R₃, or R₄ is H, hydroxyl, or its esterized or glycosylated or alkylated derivatives.

2. A method of inactivating tyrosinase activity in a subject comprising administering the patient with an effective amount of a compound of formula 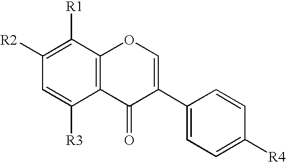
wherein R₁, R₂, R₃, or R₄ is H, hydroxyl, or its esterized or glycosylated or alkylated derivatives.

3. The method of claim 2, which is applied to whiten skin of the subject.

4. The method of claim 3, wherein the subject is a human.

5. The method of claim 4, wherein the human suffers hyperpigmentation in skin.

6. The method of claim 2, wherein the effective amount of the compound is 0.1-8.0 μM based on 0.1 μM of tyrosinase.

7. The method of claim 2, wherein the effective amount of the compound is 0.55-7.7 μM based on 0.1M of tyrosinase.
